(19) United States
(12) Patent Application Publication
(10) Pub. No.: US 2016/0193108 A1
(43) Pub. Date: Jul. 7, 2016

(54) METHOD FOR PROVIDING SKIN CARE SERVICE AND SYSTEM FOR SAME

(71) Applicant: DOCTORS TECH CO., LTD., Seongnam (KR)

(72) Inventor: Hee-Yoon CHO, Seongnam (KR)

(73) Assignee: Doctors Tech Co., Ltd., Gyeonggi-do (KR)

(21) Appl. No.: 14/442,646

(22) PCT Filed: May 13, 2013
(86) PCT No.: PCT/KR2013/004220

§ 371 Date: May 13, 2015

(30) Foreign Application Priority Data
Dec. 31, 2012 (KR) 10-2012-0158632

(51) Int. Cl. A61H 23/00; A61N 1/30; A61N 7/00; A61N 2/00; A61M 35/00

(52) U.S. Cl. CPC A61H 23/00; A61N 2/002; A61M 35/003; A61N 7/00; A61N 1/303; A61N 2007/0034

(57) ABSTRACT

The present invention relates to a skin care system and methods for providing the same, and comprises; a server including databases and a transceiver unit; a portable terminal connected wirely or wirelessly to the server, and including an application for providing skin care services; a skin care apparatus connected wirely or wirelessly to the portable terminal; and the skin care apparatus operates according to a control signal of the portable terminal, wherein the control signal is generated based on the database according to cosmetics and a user's condition provided from the server.

### TECHNICAL FIELD

The present invention relates to a method for providing skin care services and a system for the same.

### BACKGROUND ART

Cosmetics are an essential item for adult women, and vary in their types and functions. Since most of cosmetics are made for a specific effect, it may be difficult to expect other effects. Consumers purchase and use various types of cosmetics of different companies, and they need to remember all the properties and effects of cosmetic products of different companies when using them. However, there are practical difficulties to know them all to use.

In addition, the trend of cosmetic companies is to adopt methods for allowing effective ingredients of cosmetics to infiltrate into the skin with the help of external devices. That is, consumers do not stick to the method of applying different types of cosmetics by rubbing them on the skin but get the help of various devices. (For example, KR Patent No. 0537687, U.S. Pat. No. 7,282,037, JP Patent No. 4075001)

Renowned multinational cosmetic companies have been striving to develop methods for allowing effective ingredients of all their cosmetic products to infiltrate directly into the skin using a massage device.

However, the difficulties and complication of using cosmetics and devices often create a lot of confusion which keeps consumers from using them or makes them misuse methods that are against clinical and scientific evidence.

### DISCLOSURE

### Technical Problem

The present invention is devised to provide users with various cosmetics, methods for allowing skin care services (for example, massage and so on.) through proper usage of cosmetics suitable for a user's condition, and a system using thereof.

### Technical Solution

In order to solve the problems hereinbefore, a skin care system according to an exemplary embodiment of the present invention comprises: a server including databases and a transceiver unit; a portable terminal connected wirely or wirelessly to the server, and including an application for providing skin care services; a skin care apparatus connected wirely or wirelessly to the portable terminal; and the skin care apparatus operates according to a control signal of the portable terminal, wherein the control signal is generated based on the database according to cosmetics and a user's condition provided from the server.

The condition of the user may include age, skin condition, history of cosmetics use, weather conditions (temperature, the amount of ultraviolet rays, etc.) of the user's location, and so on.

The application of the portable terminal displays a control screen of the skin care apparatus, and the user can adjust the usage intensity and time of the skin care apparatus using the control screen.

The skin care apparatus may use at least one of vibration massage method, ultrasonic method, iontophoresis method, color therapy method, and magnetic therapy method for providing skin care services.

The skin care apparatus includes a vibrator which provides music and massage at the same time.

When the user uses the skin care apparatus, the application of the portable terminal transmits the history of use to the server.

The history of use stored in the server includes various users' histories that are analyzed and used to recommend proper cosmetics or skin care services to the user according to the result of the analysis.

A method for providing skin care services according to an exemplary embodiment of the present invention comprises: a step to select cosmetics from an application of a portable terminal; a step to transmit information about usage time, intensity, and instructions of a skin care apparatus suitable for the user's condition stored in the server to the portable terminal; a step to control the skin care apparatus according to a control signal of the portable terminal.

The condition of the user may include age, skin condition, history of cosmetics use, weather conditions (temperature, the amount of ultraviolet rays, etc.) of the user's location, and so on.

When a user selects cosmetics, the application of the portable terminal includes an additional step to display a control screen of the skin care apparatus, and the control screen may display information about usage time, intensity, and instructions of the skin care apparatus provided from the server.

The usage time and intensity of the skin care apparatus displayed on the control screen may be modified by the user.

The skin care apparatus may use at least one of vibration massage method, ultrasonic method, iontophoresis method, color therapy method, and magnetic therapy method for providing skin care services.

The skin care apparatus includes a vibrator, wherein a step for controlling the skin care apparatus according to a control signal of the portable terminal may allow the vibrator of the skin care apparatus to provide massage and music at the same time.

When the user uses the skin care apparatus, the application of the portable terminal may include an additional step to transmit the history of use to the server.

The history of use stored in the server includes various users' histories, and there may be an additional step to analyze and use them to recommend proper cosmetics or skin care services to the user according to the result of the analysis.

### Advantageous Effects

As described hereinbefore, using an application connected to a server of a portable terminal and a skin care apparatus, a user is provided with instructions of the skin care apparatus suitable for each cosmetic product and conditions of the user, and accordingly the user may be benefited by the effect of each cosmetic product and the effect based on their personal qualities without having particular problems. In addition, even when new cosmetics are released, the server automatically updates the information, and thus the user may be provided with skin care services applicable for the new cosmetic products.

### DESCRIPTION OF DRAWINGS

FIG. 1 is a drawing for illustrating a skin care service providing system according to an exemplary embodiment of the present invention.

FIG. 2 is an operation flow chart of an application of a portable terminal according to an exemplary embodiment of the present invention.

FIG. 3 to FIG. 7 are drawings for illustrating screens provided in an application of a portable terminal according to an exemplary embodiment of the present invention.

FIG. 8 to FIG. 10 are drawings for illustrating differences of screens by cosmetics and conditions of a user provided in an application of a portable terminal according to an exemplary embodiment of the present invention.

FIG. 11 illustrates a schematic view for collecting a user's information to the server.

FIG. 12 illustrates an example of a screen providing video contents in a portable terminal of a skin care service providing system according to an exemplary embodiment of the present invention.

### BEST MODE

With reference to the accompanying drawings, descriptions of exemplary embodiments according to the present invention are made in detail for a person having ordinary skill in the related art to easily perform. However, the present invention may be embodied in many different forms, and thus not limited to the exemplary embodiments described herein.

The thickness in drawings was enlarged in order to clearly express a plurality of layers and areas. The same reference numerals are applied to similar parts throughout the specification. When layer, film, area, plate, etc. are expressed to be “above” another part, it means that they are not only immediately above another part but also there may be other parts in the middle. On the other hand, when a part is expressed to be “immediately above” another, it means there is nothing between them.

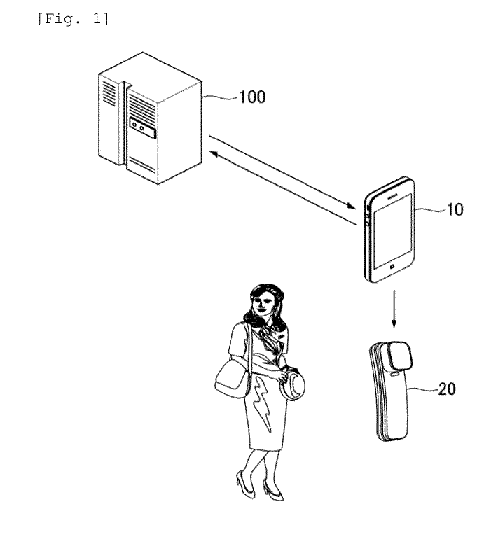

Now, a skin care service (for example, massage) providing system according to an exemplary embodiment of the present invention will be described with reference to FIG. 1.

FIG. 1 is a drawing for illustrating a skin care service providing system according to an exemplary embodiment of the present invention.

A skin care service providing system according to an exemplary embodiment of the present invention includes a server 100, a portable terminal 10, and a skin care apparatus 20. In this case, the skin care services include, for example, a massage using vibration method and others such as ultrasonic method, iontophoresis method, color therapy method, and magnetic method. Skin care services may be services to effectively infiltrate effective ingredients of cosmetics into skin. The massage using a vibration method is to put pressure to cosmetics applied to the skin for proper infiltration. In order to easily infiltrate cosmetics into skin, the ultrasonic method uses ultrasounds; the iontophoresis method uses ions; the color therapy method uses lights of a specific color (wavelength); and the magnetic therapy method uses magnetic fields.

The server 100 is connected to the portable terminal 10 through a wire or wireless communication and comprises: a first database for transmitting the information requested from the portable terminal 10; a second database for storing the information of a user using the portable terminal 10; and a transceiver unit for performing wire or wireless communications.

The first database for storing information to be provided to the portable terminal 10 includes: the information of each cosmetics; the information of massaging methods using the skin care apparatus 20; the weather conditions (temperature, the amount of ultraviolet rays, etc.) of the portable terminal's location, and so on.

The second database for storing information of a user stores their age, skin condition, and the history of use of the skin care apparatus 20.

Information of massaging methods using the skin care apparatus 20 stored in the first database may be the information about the proper intensity, usage time, and instructions of the skin care apparatus 20 based on cosmetics, users, and locations. That is, the information about massaging methods may be variable according to the weather conditions (temperature, the amount of ultraviolet rays, etc.) of the portable terminal's location, the user's age, skin condition, and the usage details of the skin care apparatus 20.

Also, the first database may store advertising contents in order for a cosmetic manufacture to have advertising effects.

The information stored in the first and the second database described hereinabove is transmitted to the portable terminal 10 by a transceiver unit. Wire and wireless communications are available in the transceiver unit, wherein sending and receiving are performed through the wireless communications according to various communication standards such as wifi, 3G, LTE communication standard, etc. The transceiver unit of the server 100 and the portable terminal 10 may be connected in the combined structure of wire and wireless communications.

The skin care apparatus 20 for the exemplary embodiment according to the present invention is an apparatus for providing massage through vibration according to a control signal of the portable terminal 10.

The skin care apparatus 20 provides massage through vibration, and other various methods such as ultrasonic method, iontophoresis method, color therapy method, magnetic therapy method, etc. may be used for providing skin care services. The skin care apparatus 20 is an apparatus that assists to effectively infiltrate effective ingredients of cosmetics into the skin and various devices may be used.

The exemplary embodiment according to the present invention mainly describes the skin care apparatus 20 (KR patent 0537687, U.S. Pat. No. 7,282,037, JP patent 4075001) using vibration for providing massage. The skin care apparatus 20 illustrated in FIG. 1 is a skin care apparatus, wherein the protruding part vibrates to provide skin massage.

The skin care apparatus 20 is connected to the portable terminal 10 through Near Field Communication, and may also be connected through wire. The Near Field Communication standard for this use has no limit, and methods including Bluetooth may be applied.

The skin care apparatus 20 operates according to a control signal from the portable terminal 10, and the operation time and intensity are controlled by the control signal of the portable terminal 10.

The portable terminal 10 may be a cellular phone and a smart phone which include an application for providing skin care services. Depending on the exemplary embodiment, the portable terminal 10 may be a PDA or a smart pad, and also be an exclusive portable terminal device in which a massage providing program is included.

Wire or wireless communication is available between the portable terminal 10 and the server 100, and wire or wireless communication (Near Field Communication) is available between the portable terminal 10 and the skin care apparatus 20.

The portable terminal 10 provides instructions (intensity, time, and methods of use) of the skin care apparatus 20 to the user based on their age, skin condition, history of use and the weather conditions (temperature, the amount of ultraviolet rays, etc.) of their locations which are provided from the server 100, and guides the user to use the skin care apparatus 20 using the methods provided through the portable terminal 10. In addition, the portable terminal 10 controls on/off modes as well as the usage intensity and time of the skin care apparatus 20. However, the user may modify the methods of use provided from the server 100 according to their preference. The targets of modification available in the application of the portable terminal 10 may be the usage intensity and time of the skin care apparatus 20.

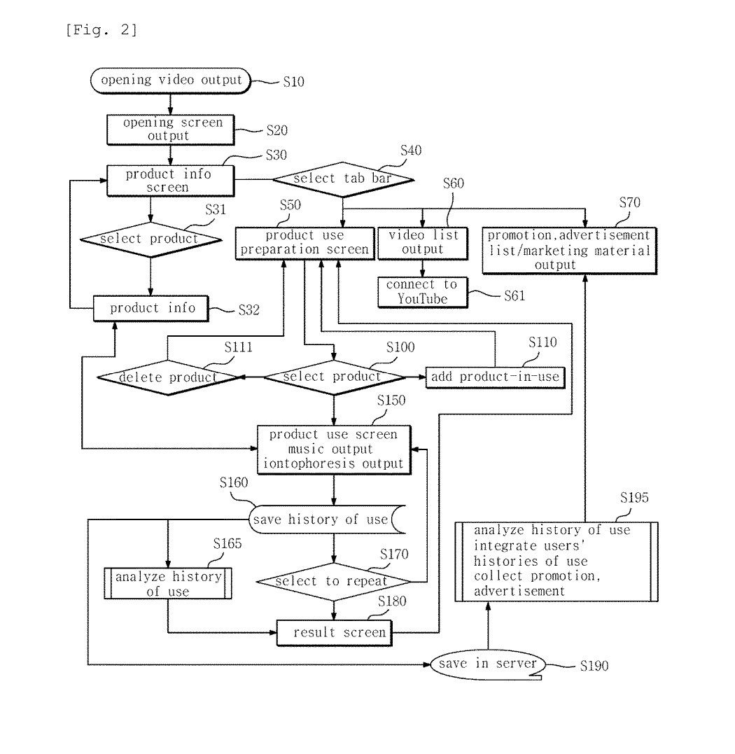

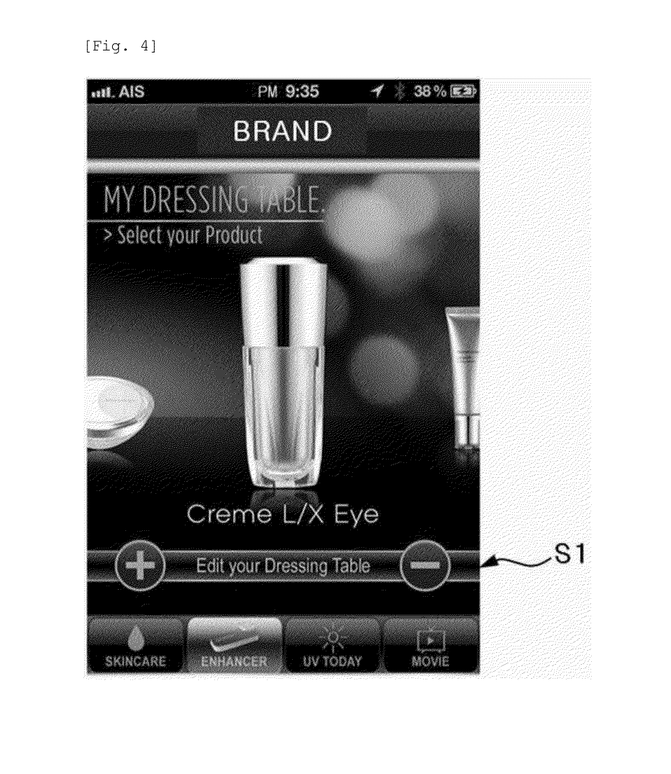

The skin care service providing application of the portable terminal 10 according to an exemplary embodiment of the present invention has an operation flow chart as illustrated in FIG. 2. And the application of the portable terminal 10 according to an exemplary embodiment of the present invention provides screens, as illustrated in FIG. 4 to FIG. 10.

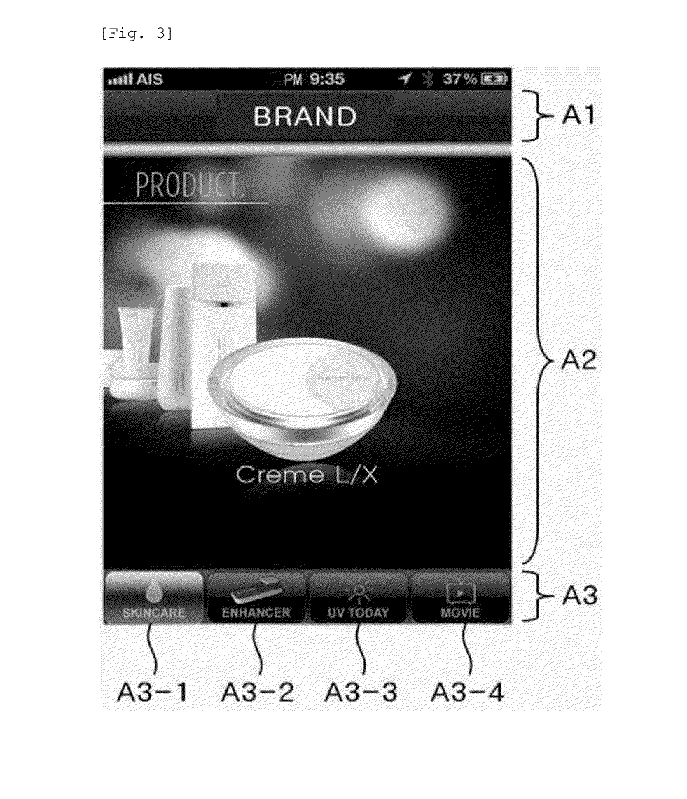

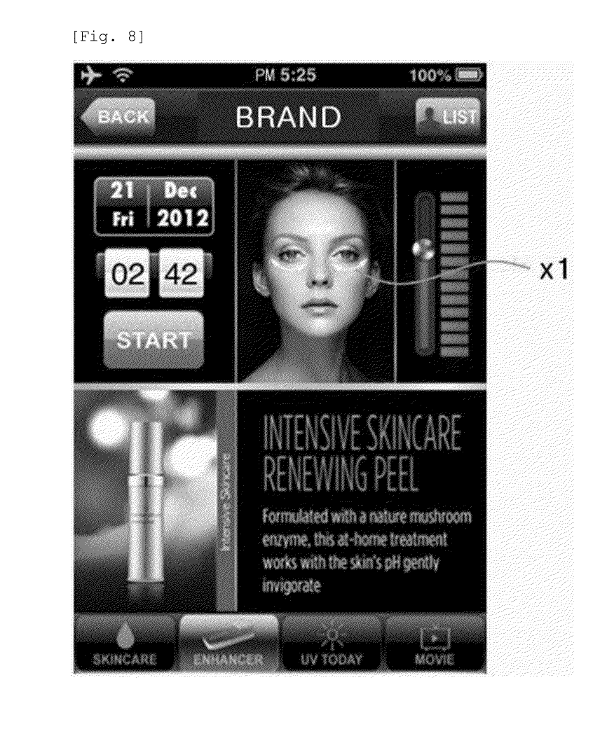

FIG. 2 is an operation flow chart of the application of a portable terminal according to an exemplary embodiment of the present invention; FIG. 3 to FIG. 7 are drawings for illustrating screens provided in an application of a portable terminal according to an exemplary embodiment of the present invention; and FIG. 8 to FIG. 10 are drawings for illustrating differences of screens by cosmetics and conditions of a user provided in an application of a portable terminal according to an exemplary embodiment of the present invention.

When a user operates an application of the portable terminal 10, a corresponding opening video of the application is output S10 for providing an opening screen S20 to the user. This step is an initial operating state of the application operation, and may be excluded from some exemplary embodiments. Information of new cosmetics, skin care information suitable for the user, advertisement of cosmetic companies, etc. may be provided to the user through this opening operation. The opening screen may be skipped or omitted by the user's operation.

After the opening screen is provided, the product information and selection screens are provided S30 for allowing the user to use the application. The product information and selection screens according to the exemplary embodiment of the present invention are illustrated in FIG. 3.

In the application according to the exemplary embodiment of the present invention, the product information and selection screens are divided into 3 regions A1, A2, A3. The A1 region displays the highest category of cosmetic products or the name of a cosmetic company. Depending on screens, nothing may be displayed or the classification of a corresponding screen may be displayed. The A2 region displays the cosmetic product's division, subcategory, name, and the shape thereof. In addition, the A2 region is the main region of the display screen and the information needs to be provided in the corresponding screen is displayed here. The A2 region may be modified by the user to display the information of different cosmetics. The A3 region divides the operation of the application according to the exemplary embodiment into largely four parts and provides buttons A3-1, A3-2, A3-3, A3-4.

A3-1 button is connected to a screen for providing information of various cosmetic products. That is, as shown in FIG. 2, when a product is selected in the step S31, a corresponding screen provides with a catalog of each product or various information about effects and ingredients of corresponding cosmetics to the user S32. Using A3-1 button, the user may obtain the information about the cosmetics they own as well as the cosmetics they will purchase.

The information of exemplary cosmetics of A3-1 button is set to the default and automatically provided to the user. The default value may be modified by the request of the server 100.

Other buttons of A3-2, A3-3, A3-4 are connected to the corresponding screens by the user's action of pressing the button.

First, take a look at A3-2 button.

When A3-2 button is selected in Tab Bar Selection step S40 of FIG. 2, a product use preparation screen is provided S50.

There is a step to select a cosmetic product S100 among various cosmetic products, wherein it may be added to or removed from the dressing table. (See FIG. 4)

FIG. 4 shows a screen, wherein the user selects a cosmetic product and adds it to the dressing table. This screen may allow the user to search the cosmetics they own and register, then include it to the dressing table. (See Step S110 in FIG. 2) This process is performed through S1 button in FIG. 4, and the S1 button is also used when the cosmetics registered in the dressing table is removed. (See Step S111 in FIG. 2)

When a registration S110 or removal S111 process of the cosmetics is completed, the screen of FIG. 4 reappears.

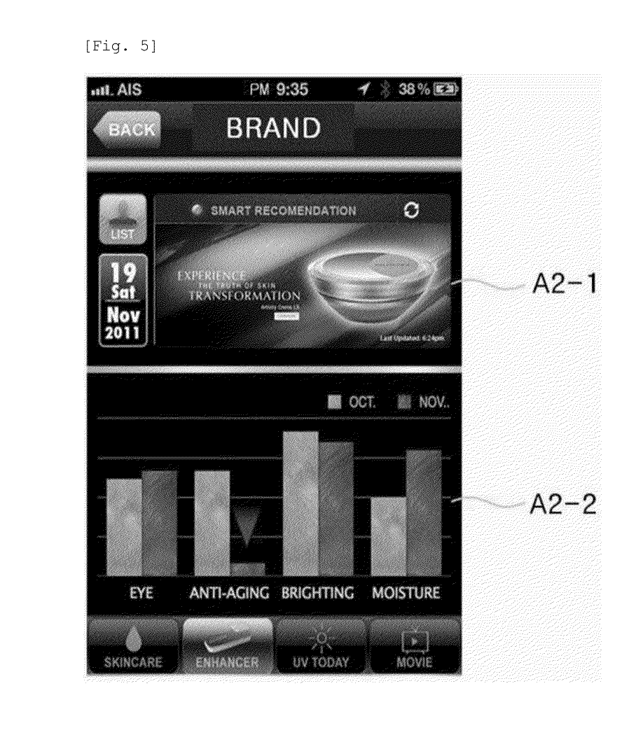

When the cosmetic product presented in the screen of FIG. 4 is selected, a screen displaying skin care services using the corresponding product is provided S150. That is, when the user wants to have the skin care service using the cosmetics presented in the screen of FIG. 4, the screen of FIG. 5 appears by clicking the photo of the cosmetics.

The screen of A2-1 in FIG. 5 includes the information of cosmetics recommended according to the status of the user (See the screen of A2-2) and its date information.

And the screen of A2-2 shows the user's skin information using a graph. The graph shows the information of eye rims skin (EYE), anti-aging (ANTI-AGING), brightening (BRIGHTENING), and moisturizing (MOISTURE). Each piece of information is displayed with two bars, wherein the left bar indicates the target value while the right bar indicates the current status of the user. The information of the user's current status may be based on the accumulated data of the user's history of cosmetics use and skin care services, which is provided in the server 100.

FIG. 5 shows the case of which the user needs to receive the skin care services using anti-aging cosmetic products.

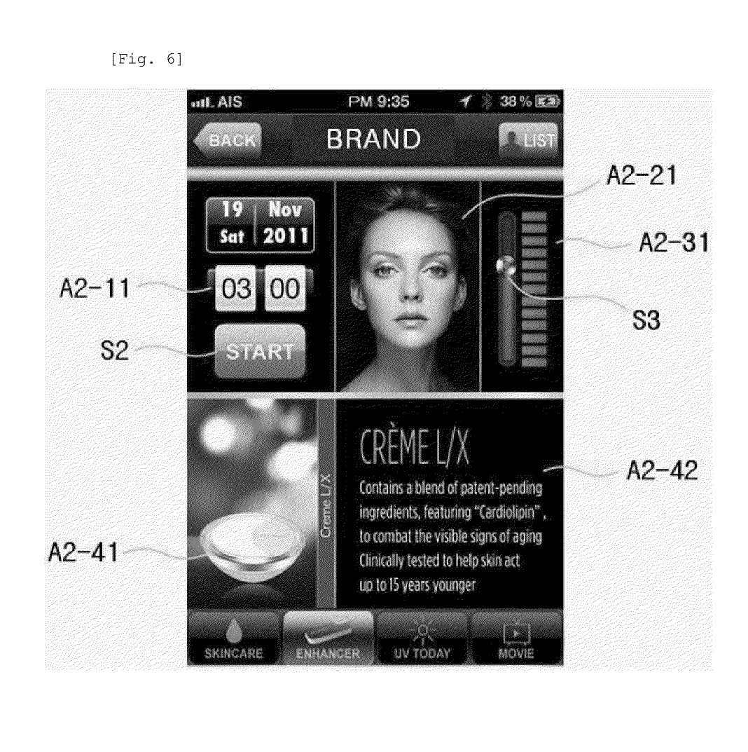

Then, as shown in FIG. 6, the user is provided with the screen for controlling the skin care apparatus 20 (hereinafter, it's also referred as the skin care apparatus control screen) and using it.

The screen of A2-11 in FIG. 6 displays date, operation time, and a Start/Stop button S2. The default operation time may be set to the operation time of the skin care apparatus 20 based on the cosmetics and the condition of the user provided from the server 100, and also be adjusted by the user operation.

The default setting value may vary according to the user's age, skin condition, history of cosmetics use, weather conditions (temperature, the amount of ultraviolet rays, etc.) of the user's location, and so on. The Start/Stop button S2 operates the skin care apparatus 20, wherein the Start button is switched to the Stop button when pressed and the operation of the skin care apparatus 20 automatically stops when the operation time is over. The user may stop the skin care apparatus 20 through additional operation even when the operation time remains.

Since the skin care apparatus 20 according to the exemplary embodiment of the present invention is devised to allow cosmetics to be more properly absorbed into the user's skin, the user is preferable to operate the skin care apparatus 20 after applying the cosmetics on the skin.

The screen of A2-21 in FIG. 6 provides with the guide about how the user moves and uses the skin care apparatus 20. The guide is not illustrated in FIG. 6, however, an exemplary embodiment of the guide is illustrated in FIG. 8 to FIG. 10 respectively.

FIG. 8 to FIG. 10 are drawings for illustrating differences of screens by cosmetics and conditions of a user provided in an application of a portable terminal according to an exemplary embodiment of the present invention.

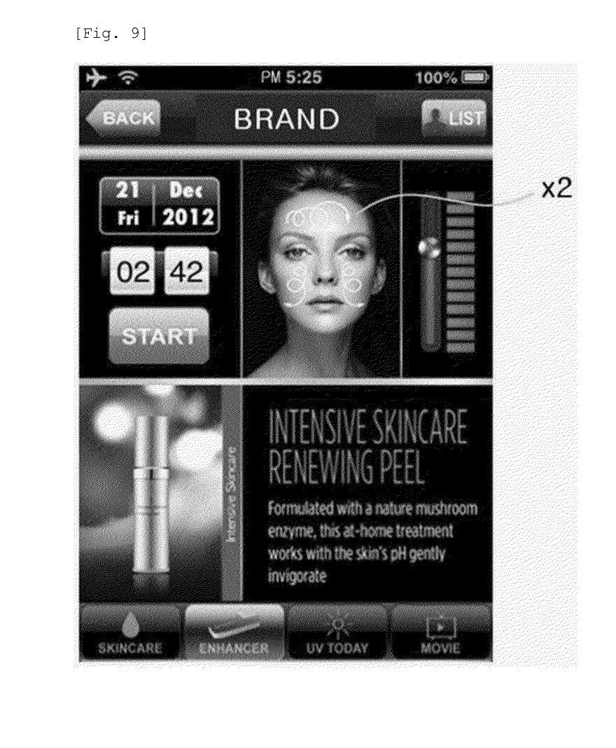

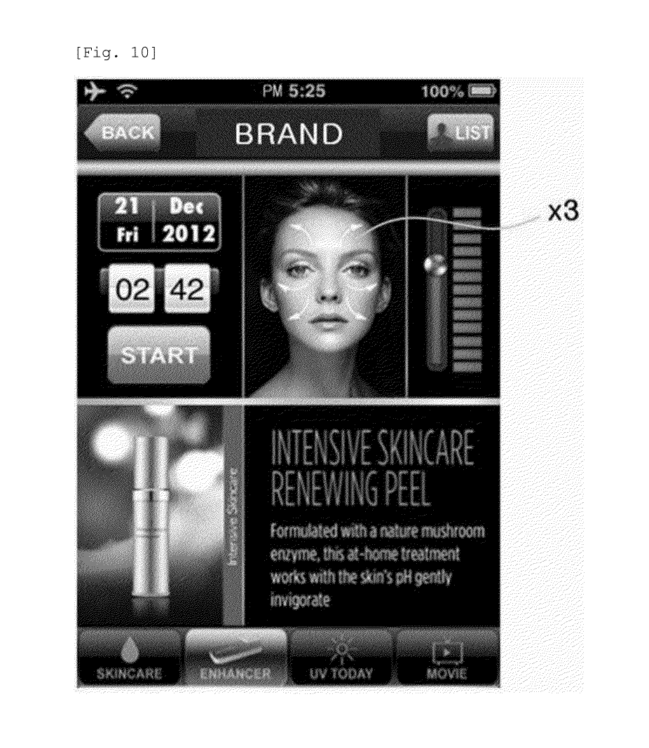

In FIG. 8, X1 arrow for guidance is marked under the eyes only; in FIG. 9, X2 arrows are circularly marked over the entire face; and in FIG. 10, X3 arrows are marked in curves towards certain directions over the entire face.

That is, the guide in FIG. 8 is provided for the case, wherein the cosmetics need to be properly absorbed into under eye area; FIG. 9 is provided for the case, wherein the cosmetics need to be evenly absorbed into the entire face; and FIG. 10 is provided for the case, wherein the cosmetics need to be absorbed into the entire face while rubbing towards the outer face.

Methods of guides may vary on cosmetics and the condition of a user, and such guides provided from the server 100 differ according to the user's location, age, condition, and the characteristics of the cosmetics.

The screen of A2-31 in FIG. 6 displays the operation intensity of the skin care apparatus 20, and e.g. in case of vibration massage, it displays the intensity of vibration. In this exemplary embodiment, the intensity is indicated by the height, and the server 100 provides the default intensity value according to cosmetics and the condition of a user. Also, the user controls the operation intensity of the skin care apparatus 20 by adjusting the position of the button S3.

The screen of A2-41 in FIG. 6 displays the cosmetics selected by the user, and A2-42 displays the description of the corresponding cosmetics. The screen of A2-42 may provide with user-specific comments according to their characteristics. The description or comments of these cosmetics may be provided from the server 100.

The step S510 of FIG. 2 outputs the screens up to FIG. 6 and operates the skin care apparatus 20 according to thereof. The skin care apparatus 20 may operate using vibration massage method as well as ultrasonic method, iontophoresis method, color therapy method, magnetic therapy, and so on.

In addition, in order to prevent the user from becoming bored during the skin care services, the vibrator of the skin care apparatus 20 may provide music. In this case, the vibrator of the skin care apparatus 20 may provide massage and music at the same time. To do that, the vibrator may separate the vibration section for music from that of massage, and vibrate alternately.

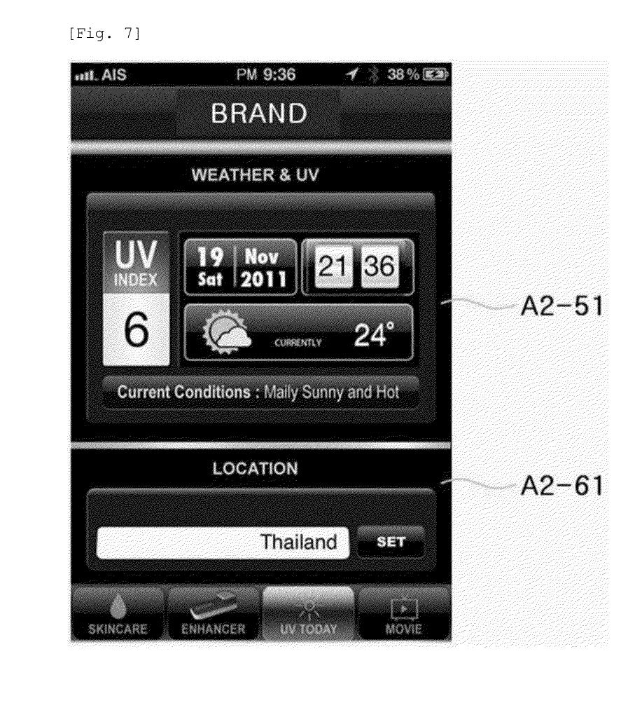

Meanwhile, when A3-3 button in FIG. 3 is pressed, the screen for providing weather and UV information follows. This is illustrated in FIG. 7, and the weather and UV information may be provided according to the local information selected by the user or automatically provided based on the GPS information of the portable terminal 10.

This UV and weather information is reflected in the server 100 and may affect the operation time and intensity of the skin care apparatus 20 that is used by the user, and becomes a standard to determine the cosmetics for recommendation to the user.

When the skin care apparatus 20 is terminated after using, the application of the portable terminal 10 stores its history of use and transmits it to the server 100 in order for it to be stored therein S190. The stored history of use is analyzed S165 in the server 100 or in the portable terminal 10, and used to determine the condition of the user's skin.

Then, there appears S170 a screen for asking to select weather the user will use the skin care apparatus 20 again or not, and when selected to use it again, the process repeats from the step S150.

When the user doesn't want to repeat the operation, the result screen of use is provided S180. The user's history of use of the skin care apparatus 20 and the result of its analysis are provided in the result screen of use.

Meanwhile, various users' histories of use stored in the server 100 may be used S195 as data for developing products and marketing for a cosmetic company.

Various users' histories of use stored in the server 100 are analyzed to output the result, and cosmetics or skin care services appropriate for a user may be recommended based on the result of corresponding analysis.

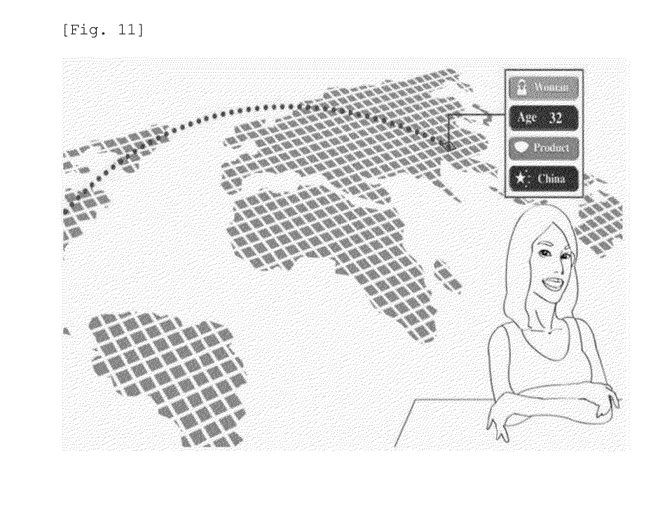

As illustrated in FIG. 11, cosmetic companies are multinational corporations and thus it's advantageous to obtain information of various weather conditions and nations as well as information for classifying preferred cosmetics by region.

The server 100 provides marketing and advertising materials again based on the user's history of use to the application of the portable terminal 10, and thus the materials may be exposed S70 to the user.

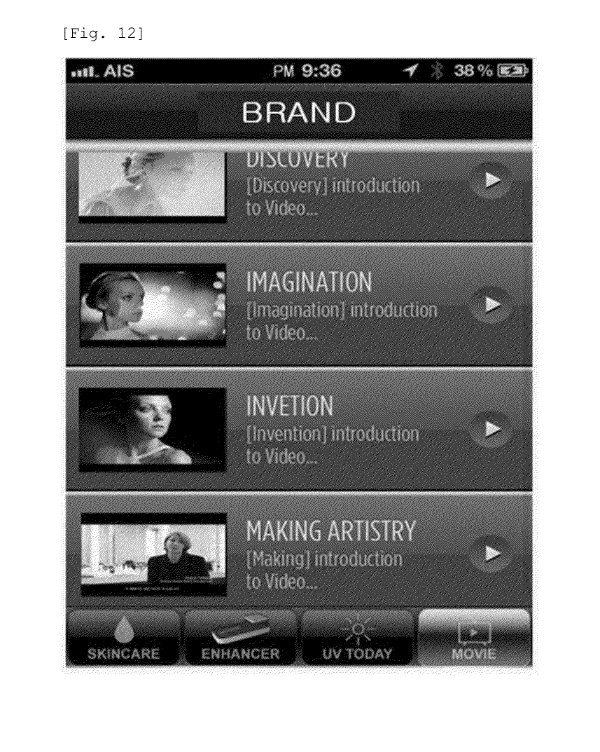

This is linked to A3-4 button of FIG. 4, and the screen provided by the A3-4 button is illustrated in FIG. 12.

As shown in the screen illustrated in FIG. 12, advertising videos and promotional videos of product characteristics are provided to the user, and it may be included in the step S60 of output video list in FIG. 2. When the user clicks a video, the corresponding video is provided S61 through web sites such as YouTube.

Various web sites other than YouTube may be used, and the videos posted in YouTube provide different videos depending on the user's age, region, and characteristics of the skin, while the order of the video list may be changed. Such information may be determined according to plural users' histories of use collected in the server 100.

FIG. 2 illustrates the detailed operation flow chart of the exemplary embodiment according to the present invention. However, in the present invention, all the procedures in FIG. 2 don't necessarily be performed in order to provide with the skin care services.

A simplified flow chart according to another exemplary embodiment of the present invention may be described hereinafter.

A user selects cosmetics in an application of a portable terminal 10, and information of the selected cosmetics in the application is transmitted to a server 100. The server 100 that received information of the selected cosmetics retransmits the information about usage time, intensity, and instructions of a skin care apparatus 20 to the portable terminal 10 based on the user's condition (the user's age, skin condition, history of cosmetics use, weather conditions (temperature, the amount of ultraviolet rays, etc.) of the user's location).

The usage time, intensity, and instructions of the skin care apparatus 20 received by the portable terminal 10 are displayed to the user through the application. And based on this, the user operates the portable terminal 10 for generating control signals of the skin care apparatus 20 in order to operate the skin care apparatus 20.

The control screen in the application of the portable terminal 10 displays the usage time, intensity, and instructions of the skin care apparatus 20, which may be provided to the user and modified by the user.

In addition, the user's history of use is transmitted to the server 100 and stored therein, while the stored history of use may be analyzed and become the basis for recommending user-specific cosmetics and skin care services.

The trend of cosmetic companies is to adopt methods for allowing effective ingredients of cosmetics to infiltrate into the skin with the help of external devices. It is difficult to infiltrate effective ingredients of cosmetics into skin only by simply rubbing the cosmetics on the skin. The present invention allows a user to properly use the skin care apparatus 20 based on their age, skin condition, history of cosmetics use, location, and the weather condition in order for effective ingredients of cosmetics to easily infiltrate into skin.

After applying the cosmetics on the skin, the user uses methods for providing skin care services and system of the same according to an exemplary embodiment of the present invention in order to properly infiltrate effective ingredients of the cosmetics into their skin and improve the effect of cosmetics use.

Moreover, the server 100 automatically transmits the information about usage time, intensity, and instructions of a skin care apparatus 20 to the portable terminal 10 based on the user's age, skin condition, history of cosmetics use, weather conditions (temperature, the amount of ultraviolet rays, etc.) of the user's location, and therefore the user is provided with professional skin care services without particular troubles.

Methods to effectively infiltrate effective ingredients of cosmetics into skin are vibration massage method, ultrasonic method, iontophoresis method, color therapy method, magnetic therapy method, and so on. The massage using a vibration method is to put pressure to the cosmetics that is applied to the skin for proper infiltration. In order for cosmetics to easily infiltrate into skin, the ultrasonic method uses ultrasounds; the iontophoresis method uses ions; the color therapy method uses lights of a specific color (wavelength); and the magnetic therapy method uses magnetic fields.

The method described hereinabove may also be used for medical apparatus, however, this exemplary embodiment of the present invention does not use the output that is the level of medical apparatus, but rather uses the lower output level. This is because when it's used by an individual without medical expertise, it may be hard on their skin. However, even though the present invention uses the skin care apparatus 20 with low output level, it may be used every time when cosmetics are used, and thus as the accumulation of usage time and frequency thereof increases, the significant effect may be expected.

This skin care service may be manipulated by a user, but since the server 100 provides with appropriate intensity, usage time, and instructions to the user according to the their age, skin condition, history of cosmetics use, weather conditions (temperature, the amount of ultraviolet rays, etc.) of the user's location, etc., the user may be conveniently provided with the skin care service appropriate for them.

The preferable exemplary embodiments of the present invention are described in detail herein, however, the protection scope of the present invention is not limited thereto, and includes a number of variations and modifications of the forms made by a person having ordinary skill in the related art using the basic concept of the present invention defined in the following scope of the claims.

1. A skin care system comprising:
a server including databases and a transceiver unit;
a portable terminal connected wirely or wirelessly to the server, and including an application for providing skin care services;
a skin care apparatus connected wirely or wirelessly to the portable terminal; and
the skin care apparatus operates according to a control signal of the portable terminal, wherein the control signal is generated based on the database according to cosmetics and a user's condition provided from the server.

2. The skin care system of claim 1,
wherein the condition of the user includes age, skin condition, history of cosmetics use, and weather condition of the user's location which is temperature or the amount of ultraviolet rays.

3. The skin care system of claim 2,
wherein the application of the portable terminal displays a control screen of the skin care apparatus, and
the user can adjust the skin care apparatus's intensity and time of use on the control screen thereof.

4. The skin care system of claim 3,
wherein the skin care apparatus provides skin care services using at least one of vibration massage method, ultrasonic method, iontophoresis method, color therapy method, and magnetic therapy method.

5. The skin care system of claim 4,
wherein the skin care apparatus includes a vibrator, and
the vibrator provides massage and music at the same time.

6. The skin care system of claim 2,
wherein when the user uses the skin care apparatus, the application of the portable terminal transmits history of use to the server.

7. The skin care system of claim 6,
wherein the history of use stored in the server includes various users' histories of use which are analyzed, and proper cosmetics or skin care services are recommended to the user according to the result of the analysis.

8. A method for providing skin care services comprising;
a step to select cosmetics in an application of a portable terminal;
a step to transmit information about usage time, intensity, and instructions of a skin care apparatus suitable for a user's condition stored in a server to the portable terminal;
a step to control the skin care apparatus by a control signal of the portable terminal.

9. The method for providing skin care services of claim 8,
wherein the condition of the user includes age, skin condition, history of cosmetics use, and weather condition of the user's location which is temperature or the amount of ultraviolet rays.

10. The method for providing skin care services of claim 9,
wherein when the user selects cosmetics, the application of the portable terminal includes an additional step to display a control screen of the skin care apparatus, and
the control screen displays information about usage time, intensity, and instructions of the skin care apparatus provided from the server.

11. The method for providing skin care services of claim 10,
wherein the usage time and intensity displayed on the control screen are indicated to be modified by the user.

12. The method for providing skin care services of claim 9,
the skin care apparatus provides skin care services using at least one of vibration massage method, ultrasonic method, iontophoresis method, color therapy method, and magnetic therapy method.

13. The method for providing skin care services of claim 12,
wherein the skin care apparatus includes a vibrator, and
a step for controlling the skin care apparatus by the signal of the portable terminal allows the vibrator of the skin care apparatus to provide massage and music at the same time.

14. The method for providing skin care services of claim 9,
wherein when the user uses the skin care apparatus, the application of the portable terminal transmits history of use to the server.

15. The method for providing skin care services of claim 14,
wherein the history of use stored in the server includes various users' histories, and there is an additional step that is to analyze and use them for recommending proper cosmetics or skin care services to the user according to the result of the analysis.
